(19) United States
(12) Patent Application Publication
(10) Pub. No.: US 2010/0233259 A1
(43) Pub. Date: Sep. 16, 2010

(54) DOSAGE FORM OF ROPINIROLE

(76) Inventors: Pascal Grenier, Kappelen (FR); Emeric Reynaud, Muttenz (CH); Alain Nhamias, Muttenz (CH); Guy Vergnault, Loechle (FR)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/637,450

(22) Filed: Dec. 14, 2009

Related U.S. Application Data
(60) Provisional application No. 61/122,076, filed on Dec. 12, 2008.

(51) Int. Cl. A61K 9/22; A61K 31/404; A61P 25/16

(52) U.S. Cl. 424/468; 514/418

(57) ABSTRACT

The present invention relates to a dosage form of Ropinirole. The dosage formulation of the invention is intended to control the release rate of one or more therapeutically active agents.

### RELATED APPLICATIONS

This application claims priority to U.S. Application No. 61/122,076 filed Dec. 12, 2008, the contents of which are incorporated by reference in their entirety.

### FIELD OF INVENTION

The present invention relates to a dosage form of ropinirole. The dosage formulation of the invention is intended to control the release rate of one or more therapeutically active agents.

### BACKGROUND OF INVENTION

Administration of biologically active agents can be in many forms and in many dosage regimens. One of the fundamental aspects of drug formulations is the targeting of a specific advantageous release profile and the targeting of a specific site of action.

There are many examples in the prior art of pharmaceutical formulations that relate to forms utilized for different administration forms, namely oral, transdermal, vaginal and ocular. The most commonly used is oral drug administration with numerous and differentiated embodiments aimed at the release of the active principle in the gastrointestinal tract. A common dosage form is a tablet or other similar small formulations, such as a capsule, which can be administered at various times of the day following a specific administration pattern or whenever desired, such as in response to sporadic symptoms such as head ache or inflammatory reaction.

Parkinson's disease (PD) is a degenerative disorder of the central nervous system that often impairs the sufferer's motor skills and speech. The condition is also characterised by muscle rigidity, tremor, a slowing of physical movement (bradykinesia), high level cognitive dysfunction, subtle language problems and, in extreme cases, a loss of physical movement (akinesia). PD can be both chronic and a progressive disease.

Ropinirole (Requip®) is known for the treatment of Parkinson's disease. The drug itself itself has a relatively short half life of around 5-6 hrs. The short half life means traditional dosage regimes of immediate release Ropinirole must be administered at regular intervals during the day. In general, the formulations have to be taken three times a day.

Moreover, it is known that dosage protocols which must be strictly adhered to can cause problems especially for individuals suffering from the progressive degeneration associated with the disease. Furthermore, patients are frequently required to take other medication concurrently which can lead to confusion and failure to adhere to the recommended dosage. Busy lives having many distractions or situations prevent an individual from following the allotted timescale for administration, which can lead to a complete lack of adherence to the regime.

Even when the administration of a drug is correctly given, i.e. at regular defined intervals during the day, there continues to be a fluctuation throughout the day in the drug concentration in the body. This fluctuation can lead to a less than optimal treatment pattern and which can then lead the patient to experience symptoms of disease. Specifically, the varying drug concentrations cause problems with dopamine receptors and can lead to the onset of L-dopa-related motor complications due to continuous dopaminergic stimulation.

As such, a once-daily formulation, 24 hour prolonged release, is advantageous because it provides controlled delivery of ropinirole over 24 hours compared to the ropinirole immediate release that must be taken three times a day resulting in fluctuations of the drug concentrations throughout the day.

### SUMMARY OF INVENTION

The invention provides a formulation system for a prolonged release of ropinirole with a therapeutically effective amount of ropinirole or salt thereof, a matrix forming agent and at least one dissolution rate controlling polymers, wherein 60-90% of the ropinirole is released by 20 hours, or more particularly between 70-90% by 20 hours. Further, in other embodiments of the invention, 0-20% of the ropinirole of salt thereof is released by 1 hour and 90-100% is released by 36 hours, or more particularly, 5-10% is released by 1 hour and between 95-100% is released by 36 hours.

In some embodiments, the formulation system of the invention comprises a monolayer tablet that is a substantial homogenous mixture of all components contained therein, for example, carboxymethylcellulose, hydrogenated castor oil, dibasic calcium phosphate and at least one diluent. The formulation system also can be a pressed coating formulation. In certain aspects of this embodiment of the invention the therapeutically effective amount of ropinirole is between about 1.0 mg and about 12.0 mg.

The invention also provides a method of making the prolonged release formulation system of the invention by providing an internal phase, wherein the internal phase comprise ropinirole or a salt thereof and a matrix forming agent, which is then sieved and mixed. An external phase is also provided containing a silicon dioxide and magnesium stearate. The external phase is then sieved and added to the internal phase, which is lubricated. The formulation is then made into a tablet using a punch press. In one embodiment of this method, the prolonged release formulation system made by this method has a dissolution rate wherein 60-90% of the ropinirole is released by 20 hours.

In another embodiment of making the formulation system of the invention, the ropinirole used in the formulation system is ropinirole hydrochloride and the internal phase is selected from carboxymethylcellulose, hydrogenated castor oil, dibasic calcium phosphate and at least one diluent.

In another embodiment of making the formulation system of the invention, the formulation system has a pressed coating. The pressed coating contains a second and third internal phase, wherein the second internal phase has a hydrogenated castor oil waxy mass and a diluent and the third internal phase is made of a silicon dioxide and magnesium stearate.

The invention also provides a method of treating Parkinson's disease with a formulation system of ropinirole is wherein a single dosage form comprising the system is administered to a subject in a 24 hour period as 60-90% of the ropinirole is released by 20 hours, or more particularly between 70-90% by 20 hours. Further, in other embodiments of the invention, 0-20% of the ropinirole of salt thereof is released by 1 hour and 90-100% is released by 36 hours, or more particularly, 5-10% is released by 1 hour and between 95-100% is released by 36 hours.

Other embodiments and features of the invention will be apparent from the following description thereof, and from the claims.

### BRIEF DESCRIPTION OF THE FIGURES

FIG. 1 is a schematic of the HPMC matrix tablet.

FIG. 2 is a schematic of the Press Coated tablet.

FIGS. 3A and 3B are schematics of an active pellet coated with a functional film.

FIG. 4 is a graph of the reference dissolution profile of ropinirole “Geomatrix®” concept.

FIG. 5 is a graph of the dissolution profiles of prototype 8200-090/08E versus ropinirole reference.

FIG. 6 is a graph showing the impact of the dosage strength (1 and 12 mg) on the dissolution profile versus the ropinirole reference.

FIG. 7 is a graph of the comparative dissolution profiles of prototype 8200-090/05Q and 8200-090/06Q versus the ropinirole reference.

FIG. 8 is a graph of the comparative dissolution profiles of prototype 8200-090/05Q versus the ropinirole reference.

FIG. 9 is a graph showing the impact of the dosage strength on the dissolution profile versus the ropinirole reference.

FIG. 10 is a graph of the comparative dissolution profiles of prototype 8200-090/02E and 8200-090/02F versus the ropinirole reference.

FIG. 11 is a graph of the comparative dissolution profiles of prototype 8200-090/02E, 8200-090/03AF/04AF and 05AF versus the ropinirole reference.

FIG. 12 is a graph of the comparative dissolution profiles of prototype 8200-090/19E-20E-21E-22E and 23E versus the ropinirole reference.

FIG. 13 is graph of the comparative dissolution profiles of prototype 8200-090/07AG1, 07AG2, 07AG3 and 07AG4 versus the ropinirole reference.

FIG. 14 is a graph of the comparative dissolution profiles of prototype 8200-090/12E and 8200-090/14E versus the ropinirole reference.

FIG. 15 is a graph of dose-normalized plasma concentration-time profiles of ropinirole 24-hour prolonged release and ropinirole immediate release in patients with early Parkinson's disease.

FIG. 16 is a graph of predicted mean concentration-time profiles (dose-normalized to 1 mg) for ropinirole 24-hour prolonged release QD and ropinirole immediate release TID (dose intervals of 6, 6, and 12 hours).

### DETAILED DESCRIPTION

The invention provides dosage forms that allow for a sustained and regular release of the active principle over a desire timescale. Preferably the dosage forms comprise a monolithic system.

Accordingly there is provided a dosage formulation comprising a therapeutically effective amount of ropinirole or salt thereof, in a form wherein the in-vitro dissolution rate of the dosage form is between 60-75% released after 20 hours and less than 8% is released at 10 hours, or more particularly the dissolution rate is:
between 0-20% released by 1 hrs;
between 60%-90% released by 20 hrs; and
between 90-100% released by 36 hrs.

Preferably, the dissolution rate is:
between 5-10% by 1 hr;
between 70%-90% by 20 hrs;
between 95%-100% by 36 hrs

The oral dosage form according to the present invention comprises any dosage form that affords the in-vitro dissolution rates within the ranges herein described and that which releases the Ropinirole in a pH interdependent manner. The oral dosage form may comprise a matrix, press coated tablet, film coated tablet, “fatty” matrix or pellets.

Preferably the dosage form will comprise a controlled release, matrix comprising one or more dissolution rate controlling polymers.

It has also been found that this sustained release product may also contribute to an improved tolerability profile and improvements in motor function and ability to perform activities during the day. The once a day formulation will also improve patients compliance with the therapy compared to the previously proposed dosage regimes.

In certain embodiments of the invention, the in vitro release rate of ropinirole is independent of pH, and to be maintained in alcohol, suggesting that the formulation is resistant to dose dumping under the conditions.

The release of a drug can be expressed by the following empirical relation:

Mₜ/M₀=Ktⁿ

The fraction of drug released (Mₜ/Mₒ) is proportional to a constant K which depends on the diffusion coefficient in the matrix, whereas the constant n depends on the swelling characteristics and the relaxation velocity of the polymeric chains and the swelling front.

According to preferred embodiments, the dosage forms of the invention are designed to allow for liberation of the active principle at a constant velocity rate, i.e. zero-order kinetics (n=0 in the above referenced formulations). In its simplest form the dosage form is an active layer comprising a hydrophilic matrix containing a drug and suitable excipients, capable of releasing the active principle at varied rates (i.e. a controlled rate of release). The effective amount of ropinirole or salt thereof is selected from 0.5 mg, 0.75 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, and 15.0 mg.

In the tablets of the present patent invention, the active substance to be carried may have a very wide solubility interval in water, e.g. between 0.01 mg/L up to 3000 g/L, preferably between 10 mg/L up to 1000 g/L (e.g. ropinirole has 133 g/L solubility), or between 0.01 mg/L up to 100 g/L.

The active substance is preferably contained in a percentage between 0.05% to 50% by weight of the active layer; more preferred ranges of the active substances are 0.05% to 40%, 0.05% to 30%, 0.05% to 10%, 0.05% to 20%.

Natural or synthetic hydrophilic polymeric substances, can be used in the preparation of said active layer which are biocompatible and/or biodegradable materials and pharmaceutically acceptable, e.g. polyvinylpyrrolidone in particular non-cross-linked polyvinylpyrrolidone (e.g. of molecular weight 30,000-400,000), hydroxypropylcellulose with a molecular weight of from 100,000 to 4,000,000, sodium carboxymethylcellulose (e.g. non-cross-linked, e.g. typical molecular weight 90,000-700,000), carboxymethylstarch, potassium methacrylate-divinylbenzene copolymer, hydroxypropylmethylcellulose with a molecular weight between 2,000 and 4,000,000, polyethyleneglycols of different molecular weights preferably between 200 and 15,000 (more preferably 1000-15,000) and polyoxyethylenes of molecular weight up to 20,000,000 (more preferably 400,000-7,000,000), carboxyvinylpolymers, poloxamers (polyoxyethylene-polyoxypropylene copolymer), polyvinylalcohols, glucanes (glucans), carrageenans, scleroglucanes (scleroglucans), mannans, galactomannans, gellans, xanthans, alginic acid and derivatives (e.g. sodium or calcium alginate, propylene glycol alginate), polyaminoacids (e.g. gelatin), methyl vinyl ether/maleic anhydride copolymer, carboxymethylcellulose and derivatives (e.g. calcium carboxymethylcellulose), ethylcellulose, methylcellulose, starch and starch derivatives, alpha, beta or gamma cyclodextrin, and dextrin derivatives (e.g. dextrin) in general. The hydrophilic polymeric substance is therefore one which can be described as a controlled release polymer or a polymeric substance which is capable of achieving controlled release (CR).

More preferably for achieving advantageous controlled release of the active substance the hydrophilic polymeric substances in the active layer comprise one or more of the following: hydroxypropylcellulose with a molecular weight of from 100,000 to 4,000, 000, hydroxypropylmethylcellulose (HPMC) with a molecular weight between 2,000 and 4,000,000 (more preferably between 10,000 and 1,500,000 molecular weight, still more preferably between 20,000 and 500,000 molecular weight, most preferably about 250,000 molecular weight), ethylcellulose or methylcellulose. The most preferred controlled release polymer is HPMC.

Hydrophilic polymeric substances such as sodium carboxymethylcellulose and/or calcium carboxymethylcellulose that act as viscosity-increasing agents/polymers or “cage-forming” components are also preferred components e.g. of the active layer. The provision of these viscosity-increasing polymers in the active layer is preferred because these help to reduce the “dose-dumping” effects occasionally seen with soluble active substances (e.g. ropinirole) whereby a significant minority of the active substance can be released from the active layer in the first hour after oral administration. Thus, it is preferred for this purpose that the hydrophilic polymeric substances in the active layer comprise sodium carboxymethylcellulose, carboxymethylcellulose or a derivative (e.g. calcium carboxymethylcellulose), hydroxypropylcellulose with a molecular weight of from 100,000 to 4,000,000, a carboxyvinylpolymer, a carrageenan, a xanthan, alginic acid or a derivative (e.g. sodium or calcium alginate, propylene glycol alginate), ethylcellulose, methylcellulose, dextrin and/or maltodextrin. Most preferred for this purpose is sodium carboxymethylcellulose (NaCMC) (e.g. non-cross-linked, e.g. typical molecular weight 90,000-700,000). The present invention also comprehends the use of other equivalent polymers able to act as viscosity-increasing agents and/or “cage-forming” components.

It is more preferred that the hydrophilic polymeric substances in the active layer comprise both the above-mentioned preferred controlled release polymers and the above-defined viscosity-increasing polymers. Thus it is preferred that the hydrophilic polymeric substances in the active layer comprise: one or more of the following components from categories (a) and (b): (a) hydroxypropylcellulose with a molecular weight of from 100,000 to 4,000,000, hydroxypropylmethylcellulose (HPMC) with a molecular weight between 2,000 and 4,000,000, ethylcellulose or methylcellulose; and (b) sodium carboxymethylcellulose, carboxymethylcellulose or derivatives (e.g. calcium carboxymethylcellulose), hydroxypropylcellulose with a molecular weight of from 100,000 to 4,000,000, a carboxyvinylpolymer, a carrageenan, a xanthan, alginic acid or a derivative (e.g. sodium or calcium alginate, propylene glycol alginate), ethylcellulose, methylcellulose, dextrin and/or maltodextrin.

The components of any formulation are important for allowing the controlled release of the active ingredient. One of the most commonly used, and most important, is the hydrophilic polymer. These can be gellable and capable of swelling upon contact with water and/or aqueous fluids, forming a gelled layer from which the active ingredient is released according to Fickian type kinetics.

In certain embodiments of the dosage forms of the invention, a matrix is covered by an impermeable barrier thereby giving impermeability and/or delaying the release of the drug carried in the matrix for a certain predetermined time period. This results in release of the active principle only from the free surface of the layer containing the active ingredient in direct contact with the dissolution medium. This type of system allows for the release of the active ingredient at a generally constant velocity.

In other embodiments of the dosage form of the invention, tablet formulations enable the release of the active principle at different release rates. This is achieved by the formulation of layers in a multi-layer tablet. Other formulations include an impermeable membrane to control the time of drug release, a complete coating of biodegradable polymeric material, a thick layer of controlled permeability materials and multilayer tablets which show a high volume increase on contact with the contents of the stomach so as to provide a prolongs gastric residence time. In addition a covering may be applied to said finished tablets by a coating process and/or any other process well known to experts in the field.

The film coating may suitably comprise a polymer. Suitable polymers will be well known to the person skilled in the art and a non-limiting list of examples include cellulose ethers, for example hydroxypropylmethyl cellulose, hydroxypropyl cellulose or methylcellulose, and copolymers of methacrylic acid and methyl methacrylate. Preferably, the film coating will comprise ethyl cellulose and methacrylic polymers.

The total film coating solids are generally applied to the solid dosage form, for example the tablet core, in an amount of from 0.5 to 10% by weight, preferably about 1 to about 5%, more preferably about 2 to about 4% based on the dry weight of the dosage form.

For example, about 6 mg of coat is applied to a tablet core weighing about 150 mg and about 9 mg of coat is applied to a tablet core weighing about 300 mg.

The film coating may additionally comprise any pharmaceutical acceptable colourants or pacifiers including water soluble dyes, aluminum lakes of water soluble dyes and inorganic pigments such as titanium dioxide and iron oxide.

The film coating may also contain one or more plasticizing agents conventionally used in polymeric film coatings, for example, polyethylene glycol, propylene glycol, dibutyl sebecate, mineral oil, sesame oil, diethyl phthalate and triacetin. Proprietary film coating materials such as Opadry, obtainable from Colorcon Ltd., UK may be used.

In certain embodiments of the invention, a functional coat is applied to the tablet cores in order to modify the release rate of the active pharmaceutical ingredient. For example, application of a coat containing polymers insoluble at low pH's (e.g. copolymers of acrylic and methacrylic acid esters) will prevent drug being released in the acidic environment of the stomach. Application of a coat containing a polymer of low aqueous solubility (e.g. ethylcellulose) may be used to modify the overall rate of drug release.

It will be appreciated that the amount of ropinirole used within the dosage form according to the present invention will be such to result in the clinically determinable improvement.

### EXAMPLES

### Example 1

### HPMC Matrix

Monolayer tablets, or a substantially homogenous tablet, were used with hydroxypropylmethylcellulose (HPMC) as matrix forming agent (E and/or K type of Methocel®), carboxymethylcellulose, hydrogenated castor oil, dibasic calcium phosphate and diluents as lactose or maltodextrin.

Blends were manufactured by dry blending. Each compound of the internal phase was sieved on a 0.710 mm screen size; a mixing step was carried out using a mixer blender for 3 min at 52 rpm and then a lubrication step was performed using a blender. Finally the external phase was sieved on a 0.500 mm screen size. A tableting step was then performed. The amount of the various ingredients in that make-up the tablet is shown in Table I below.

TABLE I

| Ingredients | mg/tb | % |
|---|---|---|
| Internal Phase | | |
| Methocel K100M | 284.16 | 63.15 |
| Ropinirole HCl | 1.14 | 0.25 |
| Lactose monohydrate | 45.00 | 10.00 |
| Blanose 9M31XF | 45.00 | 10.00 |
| Lycatab DSH | 22.50 | 5.00 |
| Cutina HR | 45.00 | 10.00 |
| External Phase | 0.00 | |
| Aerosil 200 | 2.70 | 0.60 |
| Mg Stearate | 4.50 | 1.00 |
| Total | 450.00 | 100.00 |

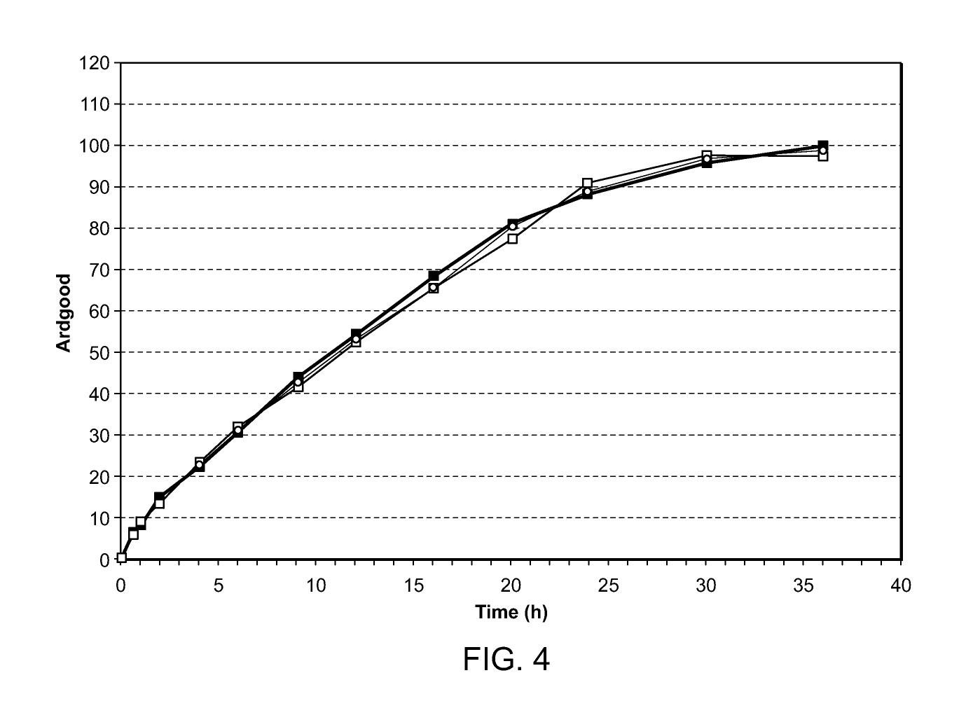

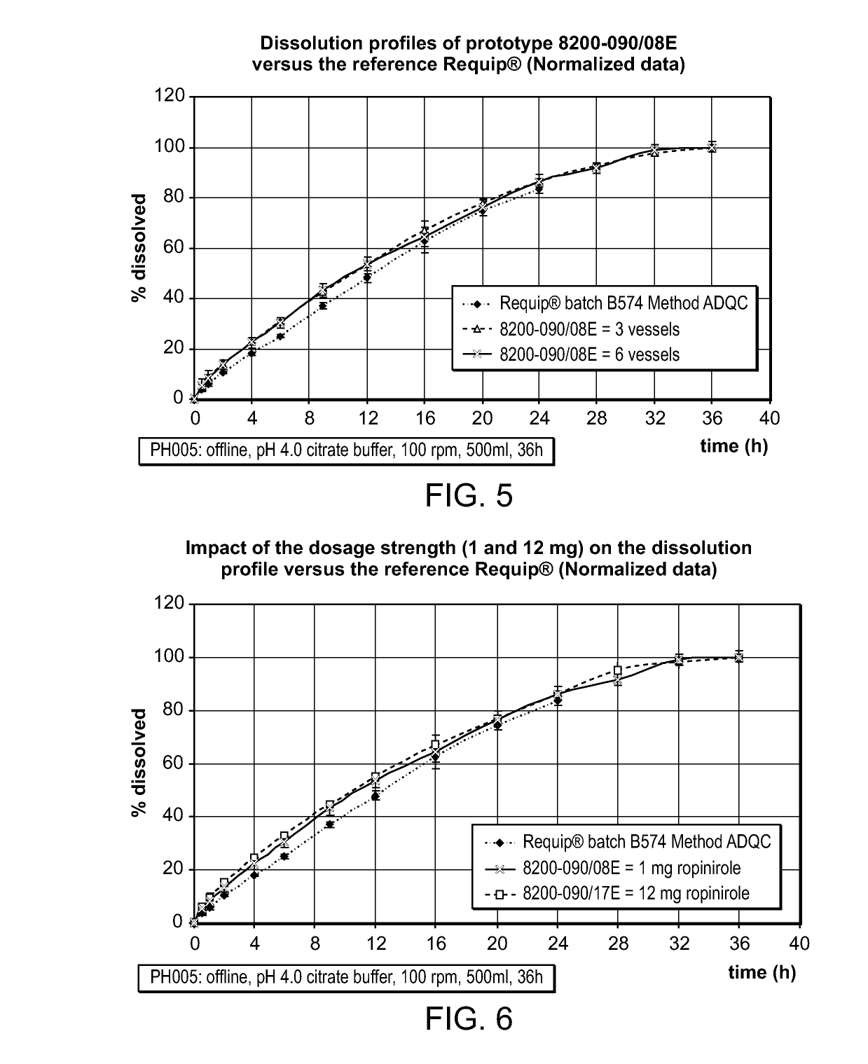

The first dissolution was performed on 3 prototypes: thickness: 6.2 mm and hardness: 150N. The second dissolution was performed on 6 prototypes: thickness: 6.3 mm and hardness: 138N. The dissolution profiles can be seen in FIGS. 5 and 6. Note that the dissolution profiles of the tablet of Example 1 matched the dissolution profile of the target profile shown in FIG. 4. In the case of the monolithic system, considering the solubility of the active pharmaceutical ingredients, it is surprising that the profile is achieved with a monolithic system, as a surface control was necessary for the multilayer tablet. Moreover with monolithic approach the same profile is achieved for both low and high dosages.

### Example 2

### Press Coating

Two tests were performed on a single punch press using the same inner core and the same outer layer blend but using two different types of tooling; a 12 mm tooling with a weight layer of 320 mg; and a 16 mm tooling with a weight layer of 500 mg.

Blends were manufactured by dry blending. Each compound of the internal phase was sieved on a 0.710 mm screen size; then a mixing step was done using a mixer blender for 3 min at 52 rpm; then a lubrication step was performed using the previous blender and the external phase was sieved on a 0.500 mm mesh screen size.

The tabletting step of the “inner cores” was performed manually using a 7 mm diameter tooling. In a second step, the final press-coated tablet was manufactured on the same single punch press using 12 mm or 16 mm diameter tooling. Details of the inner core are shown in Table II below.

TABLE II

| Ingredients | mg/tb | % |
|---|---|---|
| Internal Phase | | |
| Methocel K100M | 61.50 | 41.00 |
| Ropinirole HCl | 1.14 | 0.76 |
| Lactose monohydrate | 47.46 | 31.64 |
| Blanose 9M31XF | 15.00 | 10.00 |
| Lycatab DSH | 7.50 | 5.00 |
| Cutina HR | 15.00 | 10.00 |
| External Phase | | |
| Aerosil 200 | 0.90 | 0.60 |
| Mg Stearate | 1.50 | 1.00 |
| Total | 150.00 | 100.00 |

Physical characteristics of the inner core of the tablets were as follows:

### Inner Core:

thickness: 3.15 mm
hardness: 70N

### First Test (8200-090/05Q):

On 3 tablets: thickness: 3.9 mm
On 6 tablets: thickness: 3.9 mm
hardness: 50 N
hardness: 60 N

### Second Test (8200-090/06Q):

thickness: 3.15 mm
hardness: 50N

The details for the press coatings for the two different trials are shown in Tables III and IV below.

TABLE III
Trial N°1 (for press coating 05Q)

| Ingredients | mg/tb | % |
|---|---|---|
| Internal Phase | | |
| Cutina HR | 234.60 | 73.31 |
| Lactose | 79.00 | 24.69 |
| Internal Phase | | |
| Aerosil 200 | 3.20 | 1.00 |
| Mg stearate | 3.20 | 1.00 |
| Total | 320.00 | 100.00 |

TABLE IV
Trial N°2 (for press coating 06Q)

| Ingredients | mg/tb | % |
|---|---|---|
| Internal Phase | | |
| Cutina HR | 366.57 | 73.31 |
| Lactose | 123.43 | 24.69 |
| Internal Phase | | |
| Aerosil 200 | 5.00 | 1.00 |
| Mg stearate | 5.00 | 1.00 |
| Total | 500.00 | 100.00 |

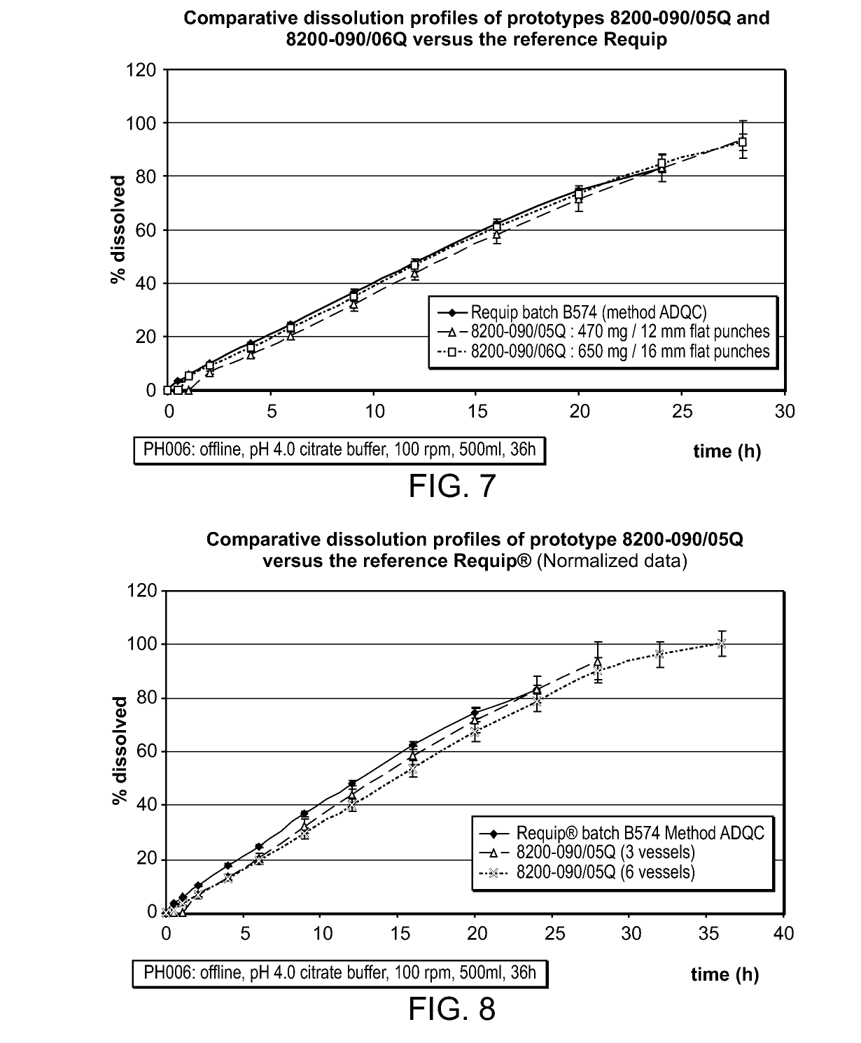

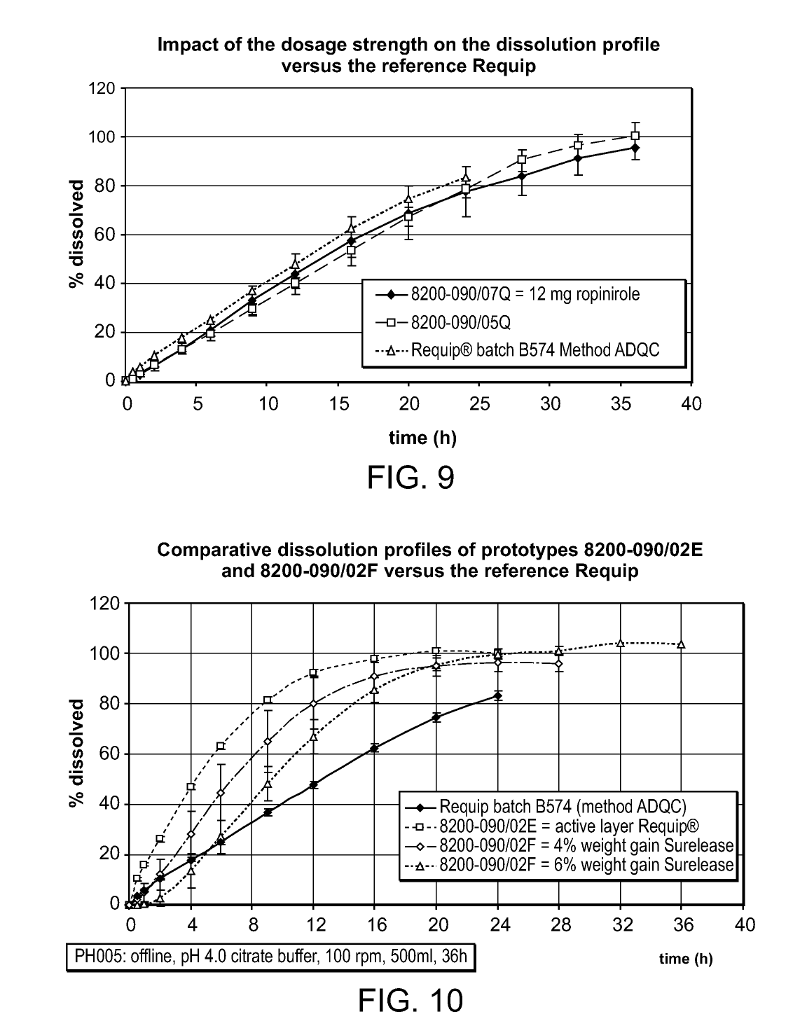

The dissolution profiles of the tablets from the different trials are shown in FIGS. 7 and 8. FIG. 9 shows the impact of the dosage strength on the dissolution profile versus the reference dissolution profile. Specifically, FIG. 9 shows the dissolution profile of a 12 mg ropinirole tablet.

Note that the dissolution profiles of the tablet of Example 2 matched the dissolution profile of the target profile in FIG. 4. It is unexpected that with the absence of active ingredient in the external mantel of the tablet to get the drug starting to be released immediately and to observe no or a minimal lag time.

### Example 3

### Film Coated Tablet

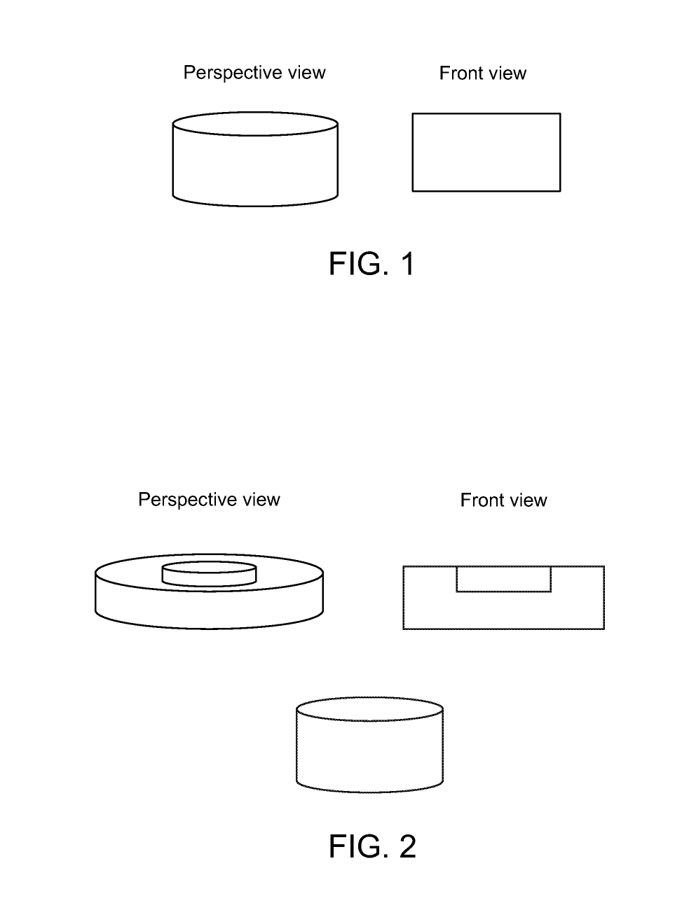

A film coating was applied to a monolayer tablet to control the active drug release rate (t_{80%}=24 hours). A pan coater (Glatt GC300 type) was used to apply the film coating. The monolayer tablets (See, FIG. 1), were manufactured by wet granulation.

The first step comprises a 50 g of lactose were weighed and sieved on a 0.710 mm mesh screen size and put into a 250 ml brown glass bottle. 2.31 g of Ropinirole (previously sieved on a 0.710 mm mesh screen size) were then added to the 45 g of sieved lactose. These materials were blended for 2 min in a tubular mixer and led to blend number 1.

The order of incorporation into the PP1 high-shear mixer was the following: Methocel, blend 1, Blanose®, Lycatab® and Cutina HR®. A four minute premix of the blend was performed at 300 rpm into the Turbula blender. Afterwards, 137 g of water was added while the impeller speed was set at 300 rpm and the chopper speed at speed 1 for 1 min. A homogenisation step was then applied using an impeller speed of 600 rpm and a chopper speed at 1 for 105 seconds.

A drying step was then implemented into the fluid bed dryer STREA-1. The inlet air temperature was set at 60° C. and the drying step was performed until the outlet temperature was 45° C. The drying step lasted 40 minutes. A milling step was then performed using the oscillating granulator (Frewitt type) at speed four. It took three minutes to mill the dry product.

The addition of the external phase was done in two steps. First with the addition of Aerosil for 2 min and then, Magnesium (Mg) Stearate was added for one minute. A Turbula blender was then used for the addition of the external phase at 74 rpm.

The blend was tableted using a Korsch® XL100 set with 8 mm round convex tooling (automatic mode) tooling machine. The pressure applied was approximately equal to 2.5 kN. The results obtained with these settings were a hardness of around 47 N and a thickness of 3.32 mm. The friability result obtained after 15 min was 0%.

A sustained release film coating was applied using a Glatt pan coater, on the finished product to control the active drug release rate to match the defined product. The control in process report is shown in Table V(a) V(b) below.

TABLE V(a)
SURELEASE FILM COATING PROCESS PARAMETERS

| | TEMPERATURE | | | | SPRAY CONTROL | | | | |
|---|---|---|---|---|---|---|---|---|---|
| | OUTLET | INLET | | BLOW | AIR | | | | PAN |
| TIME | REAL | REAL | PROG | SPEED | PRESSURE | PUMP | | | SPEED |
| min | ° C. | ° C. | ° C. | m3/h | dbar | % | g of FC | g/min | rpm |
| 13 h 35 | 45 | 54 | 55 | 110 | — | — | — | — | 2 |
| 13 h 45 | 38 | 55 | 57 | 117 | 25 | 5 | 100 | 12 | 10 |
| 13 h 50 | 39 | 56 | 57 | 121 | 25 | 5 | 150 | 11.5 | 10 |
| 14 h 10 | 40 | 56 | 57 | 120 | 25 | 5 | 360 | 11.5 | 10 |
| 14 h 25 | 40 | 57 | 57 | 120 | 25 | 5 | 540 | 11.5 | 10 |
| 14 h 40 | 41 | 57 | 57 | 120 | 25 | 5 | 720 | 11.5 | 10 |
| 14 h 55 | 41 | 57 | 57 | 118 | 25 | 5 | 900 | 11.5 | 10 |
| 15 H 05 | 41 | 57 | 57 | 118 | 25 | 5 | 900 | 11.5 | 10 |
| 15 h 08 | 50 | 57 | 57 | 123 | — | — | — | — | 5 |

TABLE V(b)
EUDRAGIT RL/RS30 PROCESSING PARAMETERS

| | TEMPERATURE | | | | SPRAY CONTROL | | | | |
|---|---|---|---|---|---|---|---|---|---|
| | OUTLET | INLET | | BLOW | AIR | PUMP | | | PAN |
| TIME | REAL | REAL | PROG | SPEED | PRESSURE | | g of | | SPEED |
| min | ° C. | ° C. | ° C. | m3/h | dbar | % | FC | g/min | rpm |
| 10 h 50 | 39 | 45 | 45 | 120 | — | — | — | — | 6 |
| 11 h | 34 | 45 | 45 | 120 | 20 | 5 | 56 | 14 | 10 |
| 11 h 09 | 31 | 50 | 50 | 120 | 20 | 5 | 191 | 14 | 10 |
| 11 h 20 | 32 | 49 | 48 | 120 | 20 | 5 | 378 | 14 | 10 |
| 11 h 35 | 31 | 48 | 48 | 120 | 20 | 5 | 565 | 14 | 10 |
| 11 h 47 | 31 | 48 | 48 | 120 | 20 | 5 | 750 | 14 | 10 |
| 12 h | 31 | 48 | 48 | 120 | 20 | 5 | 938 | 14 | 10 |
| 12 h 03 | 41 | 48 | 48 | 120 | — | — | — | — | 6 |

The qualitative and the quantitative formulation of the active layer is shown in Table VI below.

TABLE VI

| Ingredients | mg/tb | % |
|---|---|---|
| Internal Phase | | |
| Methocel K100M | 61.50 | 41.00 |
| Ropinirole HCl | 1.14 | 0.76 |
| Lactose monohydrate | 47.46 | 31.64 |
| Blanose 9M31XF | 15.00 | 10.00 |
| Lycatab DSH | 7.50 | 5.00 |
| Cutina HR | 15.00 | 10.00 |
| External Phase | | |
| Aerosil 200 | 0.90 | 0.60 |
| Mg Stearate | 1.50 | 1.00 |
| Total | 150.00 | 100.00 |

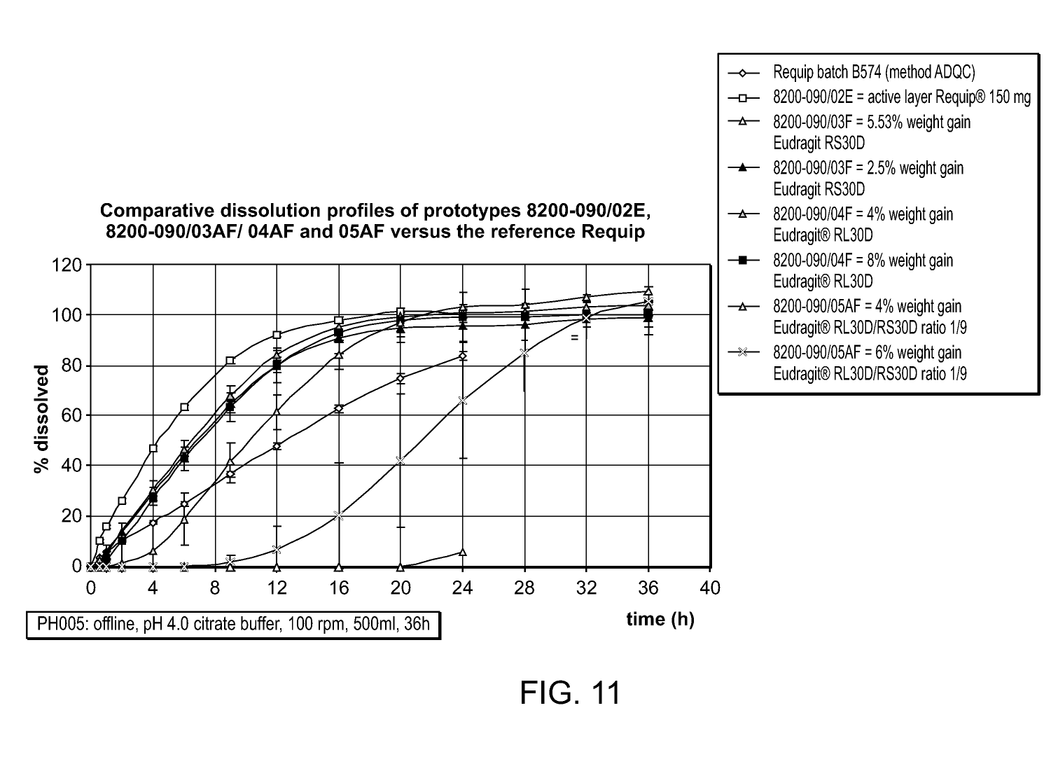

A graph showing the comparative dissolution profiles of the tablets of Example 3, the ropinirole core, and the reference ropinirole tablet is shown in FIG. 10. Specifically, the impact of the film-coating, i.e. weight gain 4% and 6%, on the tablet is shown. Further, FIG. 11 shows the impact of various percentages of film applied and the impact of the percent weight gain on the dissolution profile.

### Example 4

### “Fatty” Matrix

Monolayer “fatty” matrix tablets using different types of lipidic compounds, such as Compritol 888 ATO®, Cutina HR®, were also manufactured by direct compression and tableted on a Korsch EKO using 10 mm flat punches. Blends were manufactured by dry blending. Each compound of the internal phase was sieved on a 0.710 mm screen size; a mixing step was done using a mixer blender (Turbula type) for 3 minutes at 52 rpm. A lubrication step was performed using the previous blender and the external phase was sieved on a 0.500 mm mesh screen size.

### Examples of Fatty Matrix Tested:

Cutina HR/Lactose pulvis
Cutina HR/Avicel PH102
Cutina HR/HPMC (Methocel E4M, Methocel K4M, Methocel K100M . . . )
Cutina HR/Avicel PH102/Encompress

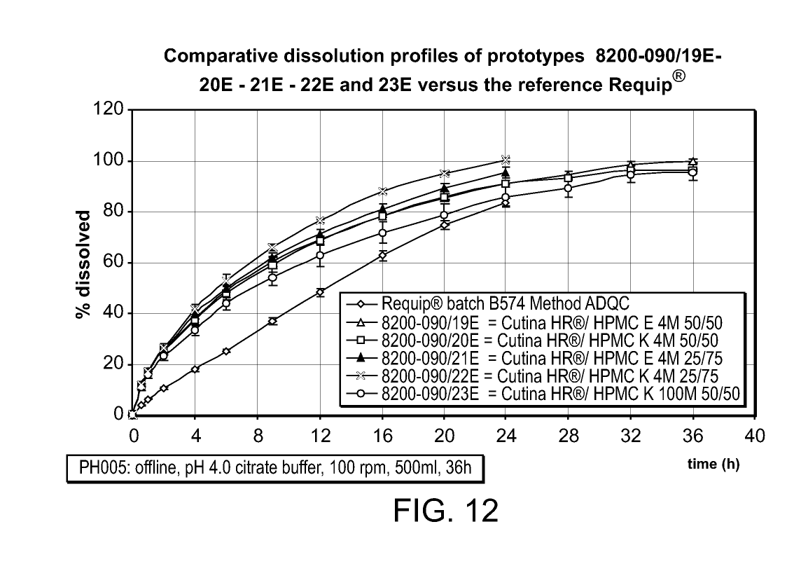

The dissolution profiles are shown in FIG. 12. For this concept, the last prototype 8200-090/23E gave an in-vitro profile which was very close to the reference profile.

### Example 5

### Pellets

Pellets of Ropinerole on which a functional film coating was applied to control the active drug release rate and to match the defined target (t_{80%}=24 hours). The final galenical dosage form could be a tablet, a capsule or a sachet.

The drug loaded pellets (Cellets® or Celphere® CP507) and the film coating process were implemented in a fluid bed dryer (Glatt® GPCG1).

The first step included a binding solution containing the API and PVP (Polyvinylpyrrolidone) that was sprayed on microcrystalline cellulose pellets to stick the active drug onto the pellets. The ingredients of the two different types of pellets are shown in Tables VII and VIII below.

TABLE VII
Active coating formulation: 09AF2

| INGREDIENTS | THEORETICAL | |
|---|---|---|
| (cellets ® 1 000) | WEIGHT (g) | (%) |
| Ropinirole HCL | 5.70 | 1.9% |
| PVP | 3.00 | 1.0% |
| Water | 291.30 | 97.1% |
| TOTAL | 300.00 g | 100.0% |

TABLE VIII
Active coating formulation: 06AF

| INGREDIENTS | THEORETICAL | |
|---|---|---|
| (Celphere ® CP507) | WEIGHT (g) | (%) |
| Ropinirole HCL | 3.8 | 1.9 |
| PVP | 5.00 | 2.5 |
| Water | 191.2 | 95.6 |
| TOTAL | 200 | 100.0 |

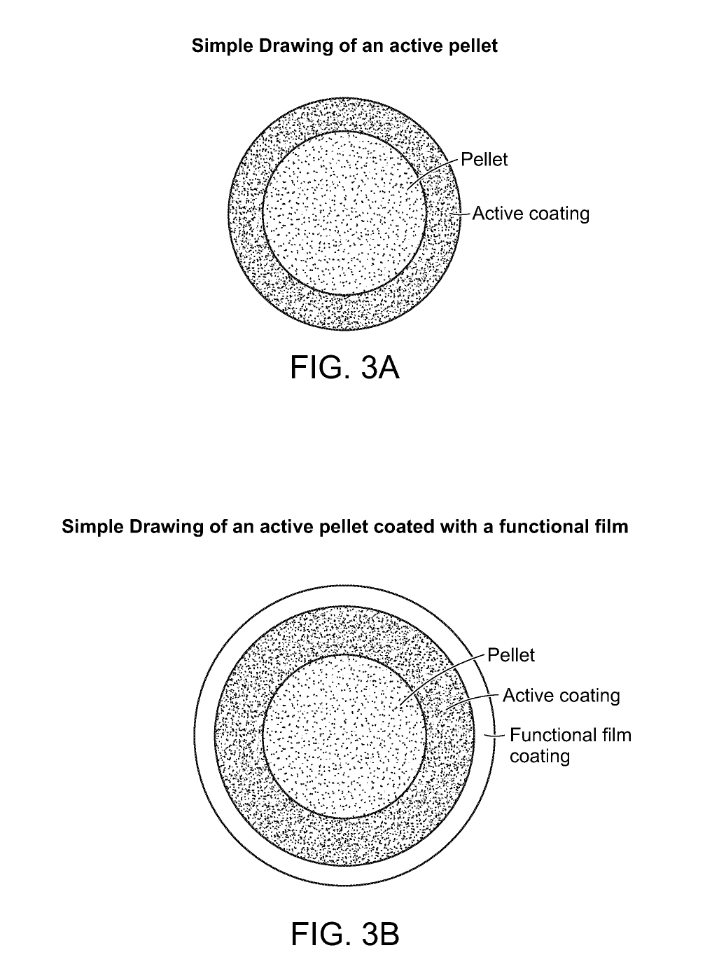

A functional film coating was then applied on drug loaded pellets to control the active drug release rate. (See, FIG. 3A). The ingredients comprising the film coatings are shown in Tables IX and X.

TABLE IX
Functional film-coating formulation:
10AF1 (Eudragit film coated pellets)

| INGREDIENTS | THEORETICAL | |
|---|---|---|
| (on 09AF2) | WEIGHT (g) | (%) |
| Eudragit RL30D | 39.3 | 3.93 |
| Eudragit RS30D | 353.2 | 35.32 |
| Triethylcitrate | 23.3 | 2.33 |
| Talc | 59 | 5.90 |
| Water | 525.2 | 52.52 |
| Total | 1000 | 100.0% |

TABLE X
Functional film-coating formulation:
08AF (Surelease film coated pellets)

| INGREDIENTS | THEORETICAL | |
|---|---|---|
| (on 06AF) | WEIGHT (g) | (%) |
| Surelease E7 19010 | 600 | 60 |
| Water | 400 | 40 |
| Total | 100 | 100 |

A schematic of the pellet after the film-coating is applied is shown in FIG. 3B.

The third step included filling the capsules, a clear/clear Licaps® size 1 was used. Each capsule was filled with the film-coated pellets at different weight gains as shown in Table XI below.

TABLE XI

| % weight | 10AF1 | | | |
|---|---|---|---|---|
| gained | 5 | 10 | 15 | Aerosil 200 |
| 07AG1 (mg) | 132 | | | no |
| 07AG2 (mg) | | | 153 | no |
| 07AG3 (mg) | | 142.7 | | no |
| 07AG4 (mg) | | | 153 | 0.8 |

Two of the prototypes in Table XII, were tableted as monolayer tablets manufactured manually on a single press.

TABLE XII

| Prototypes | 08AF (pellets) | Support layer blend |
|---|---|---|
| 8200-090/12E | 210 mg | 210 mg of 1029/05B1 |
| | | (D2 Methocel K15M) |
| 8200-090/14E | 210 mg | 210 mg of 9990/01B |
| | | (D1 Methocel K100M) |

For each prototype, a blend was made (before tabletting step) using a Turbula blender. The blend was a mixture between pellets (08AF) and the support layer blend.

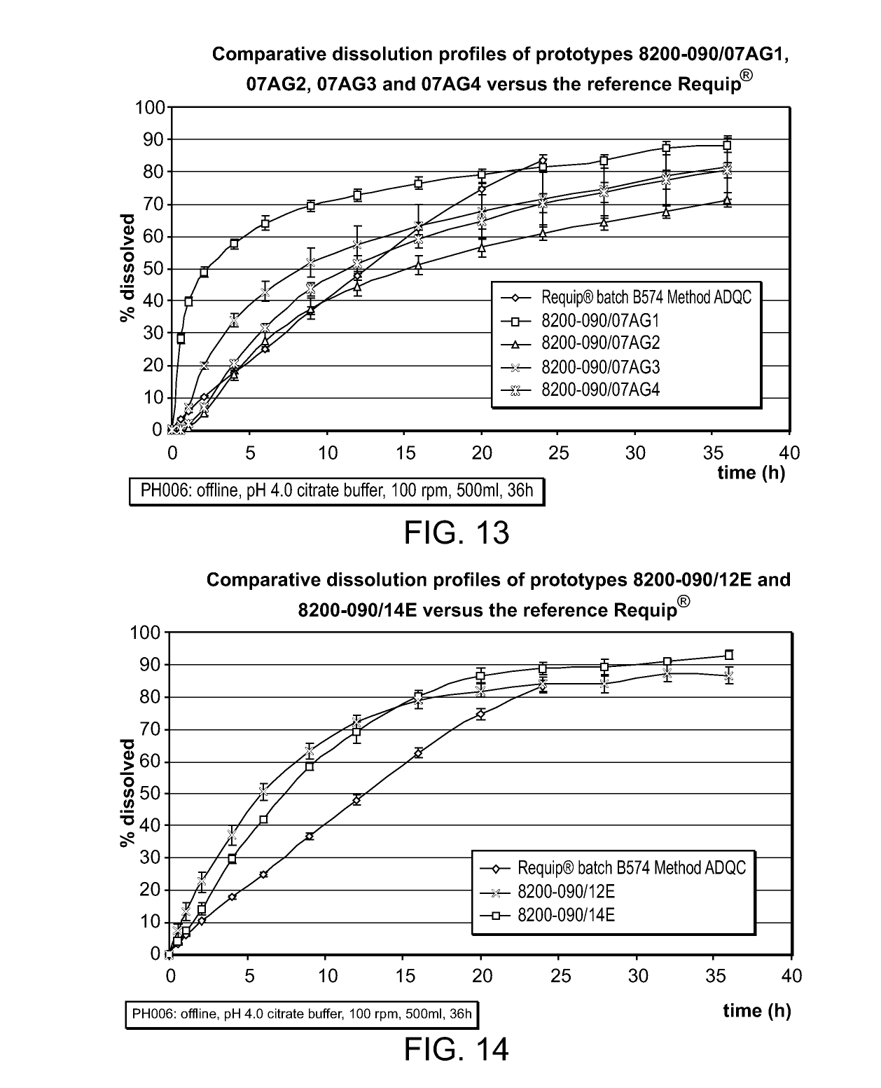

The dissolution profiles of the pellets and the tablets are shown in FIGS. 13 and 14.

### Example 6

A study was conducted to characterize the steady-state pharmacokinetics of ropinirole 24-hour prolonged release in patients with Parkinson's disease. The study was a 2 part study that employed a crossover design to assess the relative bioavailability of steady state ropinirole 24-hour prolonged release 8.0 mg daily and ropinirole immediate release 2.5 mg, taken three times daily. The second aspect of the study evaluated the effect of food intake on the rate and extent of ropinirole absorption from ropinirole 24-hour prolonged release 8 mg QD. The study assessed the dose proportionality of ropinirole 24-hour prolonged release 2-, 4-, and 8-mg tablets and the dose-strength equivalence of four 2-mg tablets compared with one 8-mg tablet. Intensive pharmacokinetic blood sampling was performed over 24 hours. Steady-state Cₘᵢₙ, Cₘₐₓ, AUC from time zero to 24 hours after dosing (AUC₀_{_{—}}₂₄), and Tₘₐₓ were determined by non-compartmental methods. The 24-hour release prolonged release tablets comprise the formulation disclosed herein.

Ropinirole 24-hour prolonged release provided continuous delivery of ropinirole over 24 hours, resulting in a smooth plasma concentration-time profile, and food had no significant effect on absorption. Dose-normalized AUC₍₀₋₂₄₎ and C₍ₘᵢₙ₎ were similar for both formulations, and dose-normalized C₍ₘₐₓ₎ was slightly lower for ropinirole 24-hour prolonged release. The relative bioavailability data indicated that patients may switch overnight from ropinirole immediate release to ropinirole 24-hour prolonged release while maintaining similar daily exposure. The pharmacokinetics of ropinirole were dose proportional over the range from 2 to 8 mg. The dose strengths of four 2 mg tablets and one 8-mg tablet of ropinirole 24-hour prolonged release were found to be equivalent.

Studies have evaluated the relative bioavailability of ropinirole 24-hour prolonged release and ropinirole immediate release to help determine the strategy for switching patients from the immediate-release formulation to the 24-hour prolonged-release formulation, and evaluations of food effects, dose proportionality, and dose-strength equivalence of ropinirole 24-hour prolonged release.

On a dose-for-dose basis, AUC₀₋₂₄ and Cₘᵢₙ values were similar for ropinirole 24-hour prolonged release QD and ropinirole immediate release TID. However, Cₘₐₓ values were 12% lower for ropinirole 24-hour prolonged release than for ropinirole immediate release. Patients who received ropinirole immediate release (2.5 mg TID) were able to switch overnight to the closest dose of ropinirole 24-hour prolonged release (8 mg QD), with no effect on the tolerability profile. This finding was supported by data from the EASE-PD Monotherapy study in which a population pharmacokinetic analysis indicated that on average, systemic exposure to ropinirole was similar when patients switched from ropinirole immediate release to ropinirole 24-hour prolonged release (based on the nearest total daily dose), with no clinically significant differences in efficacy or tolerability between the 2 formulations.

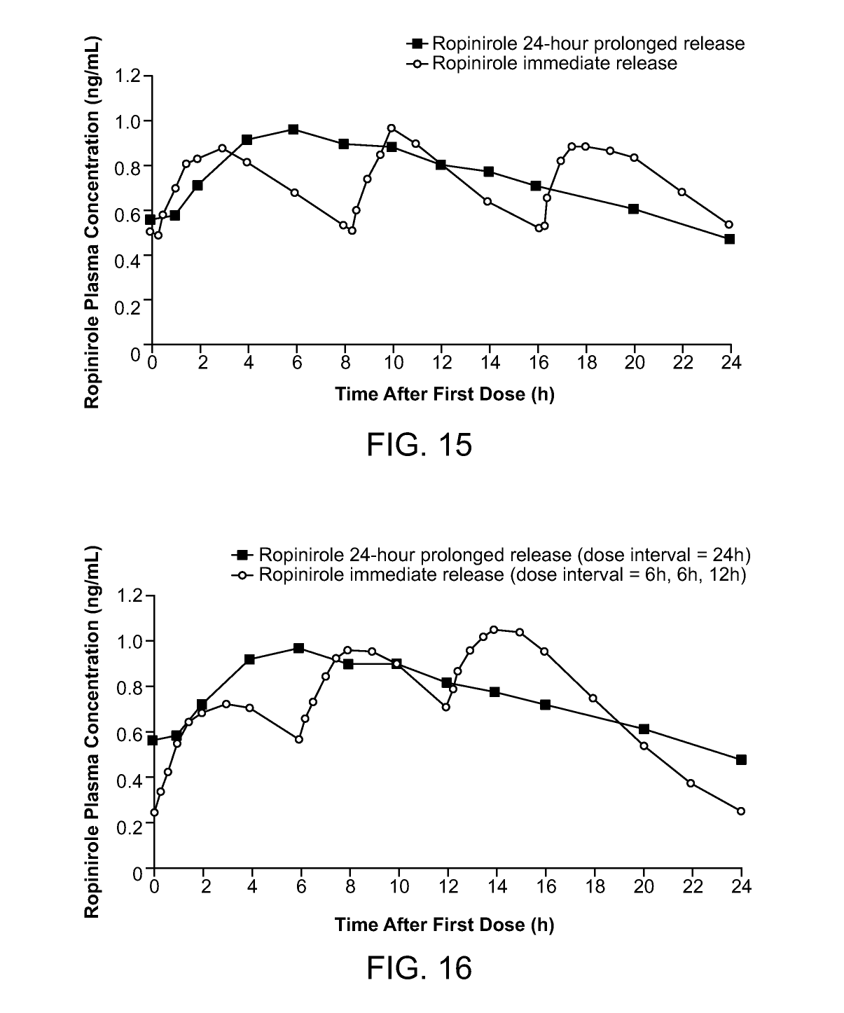

The mean degree of fluctuations for ropinirole 24-hour prolonged release was 0.66 and for ropinirole immediate release was 0.85, indicating that there was slightly lower variation in the range of ropinirole concentrations over a 24-hour period with ropinirole 24-hour prolonged release QD compared with ropinirole immediate release taken three times daily. (See, FIG. 15). As it is recommended that ropinirole immediate release be taken with meals, it appears likely that in clinical practice, ropinirole immediate release would be taken at breakfast, lunch, and dinner (e.g., at 7:00 AM, 1:00 PM, and 7:00 PM), approximating a 6-, 6-, and 12-hour dosing regimen. This dosing regimen would be predicted to result in much larger fluctuations in plasma concentrations over a 24-hour period compared with ropinirole 24-hour prolonged release. (See, FIG. 16). With a 6-, 6-, and 12-hour regimen of ropinirole immediate release, there is −5-fold fluctuation between the maximum and minimum ropinirole concentrations, compared with a 2-fold fluctuation with ropinirole 24-hour prolonged release. Patients taking this regimen of ropinirole immediate release would, therefore, be predicted to have a 2-fold lower concentration of ropinirole first thing in the morning compared with patients taking ropinirole 24-hour prolonged release. The once-daily dosing regimen of ropinirole 24-hour prolonged release is associated with a reduced frequency of fluctuations compared with ropinirole immediate release. (See, FIGS. 15 and 16).

There is no clinically relevant effect of food on the pharmacokinetics of ropinirole after administration of ropinirole 24-hour prolonged release in patients with PD. Values for ropinirole 24-hour prolonged release were similar in both the fed and fasted states. The Cₘₐₓ for ropinirole 24-hour prolonged release was higher (15%) and Tₘₐₓ delayed (−2 hours) in the fed state compared with the fasted state. This small increase in Cₘₐₓ and delay to Tₘₐₓ are not clinically relevant due to the relatively flat concentration-time profile of ropinirole after administration of ropinirole 24-hour prolonged release. In addition, there was no evidence to suggest that the controlled release of ropinirole from the 24-hour prolonged-release formulation was affected by co-administration with food, and, therefore, dose dumping would not be expected to occur. These results suggest that ropinirole 24-hour prolonged release can be taken without regard to food intake.

The similarity in pharmacokinetics of the multiple- and single-tablet regimens was also had similar incidences of adverse events. Therefore, patients beginning treatment with ropinirole 24-hour prolonged release on a regimen of multiple low-dose tablets (four 2-mg tablets daily) can switch to an equivalent single dose of ropinirole 24-hour prolonged release (one 8-mg tablet daily) and maintain systemic exposure without any clinically relevant effects on tolerability.

In patients with PD, ropinirole 24-hour prolonged release provided continuous delivery of ropinirole over 24 hours, resulting in a smooth pharmacokinetic profile with reduced fluctuations in plasma levels over 24 hours compared with ropinirole immediate release. Systemic exposure was similar between ropinirole immediate release and ropinirole 24-hour prolonged release based on the same total daily dose, indicating that patients may switch overnight from the immediate-release formulation to a similar total daily dose of the prolonged-release formulation while maintaining similar daily exposure. Food was found to have no clinically relevant effect on absorption, and the pharmacokinetics of ropinirole were dose proportional over the dose range from 2 to 8 mg. Furthermore, patients taking multiple low-dose tablets (four 2-mg tablets) were able to switch to a single higher-dose tablet (8 mg) without a reduction in systemic exposure.

What is claimed is:

1. A formulation system for a prolonged release of ropinirole comprising a therapeutically effective amount of ropinirole or salt thereof, a matrix forming agent and at least one dissolution rate controlling polymers, wherein the formulation has a dissolution rate wherein 60-90% is released by 20 hours.

2. The formulation system of claim 1, wherein the dissolution rate of the ropinirole or salt thereof is between 90-100% by 36 hours.

3. The formulation system of claim 1, wherein the formulation has a dissolution rate wherein 0-20% of the ropinirole of salt thereof is released by 1 hour.

4. The formulation system of claim 3, wherein the dissolution rate of the ropinirole or salt thereof is between 5-10% by 1 hour.

5. The formulation system of claim 2, wherein the formulation has a dissolution rate wherein 95-100% is released by 36 hours.

6. The formulation system of claim 1, wherein the dissolution rate of the ropinirole or salt thereof is between 70-90% by 20 hours.

7. The formulation system of claim 1, wherein the formulation system comprises a monolayer tablet wherein the monolayer tablet comprises a substantially homogenous mixture of all components.

8. The formulation system of claim 1, wherein the formulation further comprises carboxymethylcellulose, hydrogenated castor oil, dibasic calcium phosphate and at least one diluent.

9. The formulation system of claim 1, wherein the formulation system comprises a pressed coated formulation.

10. The formulation system of claim 1, wherein the therapeutically effect amount of ropinirole is about 1.0 mg.

11. The formulation system of claim 1, wherein the therapeutically effect amount of ropinirole is about 12.0 mg.

12. A method of making a formulation system for the prolonged release of ropinirole comprising:
providing an internal phase, wherein the internal phase comprise ropinirole or a salt thereof and a matrix forming agent;
sieving the internal phase;
mixing the internal phase;
providing an external phase, wherein the external phase comprises a silicon dioxide and magnesium stearate;
sieving the external phase;
lubricating the internal phase; and
tableting the formulation with a punch press
wherein the formulation has a dissolution rate wherein 60-90% is released by 20 hours.

13. The method of making the formulation of claim 12, wherein the ropinirole is ropinirole hydrochloride.

14. The method of making the formulation of claim 13, wherein the internal phase further comprising carboxymethylcellulose, hydrogenated castor oil, dibasic calcium phosphate and at least one diluent.

15. The method of making the formulation of claim 12, further comprising a pressed coating, wherein the pressed coating comprises a second and third internal phase, wherein the second internal phase comprises a hydrogenated castor oil waxy mass and a diluent and the third internal phase comprises a silicon dioxide and magnesium stearate.

16. A method of treating Parkinson's Disease comprising administering to a subject in need thereof the formulation system of a prolonged release of ropinirole comprising a therapeutically effective amount of ropinirole or salt thereof, a matrix forming agent and at least one dissolution rate controlling polymers, wherein the formulation has a dissolution rate wherein 60-90% is released by 20 hours.

17. The method of treating of claim 16, wherein the subject is administered a single formulation in a 24 hour period.

18. The method of treating of claim 16, wherein the dissolution rate of the ropinirole or salt thereof is between 90-100% by 36 hours.

19. The method of treating of claim 16, wherein the formulation has a dissolution rate wherein 0-20% of the ropinirole of salt thereof is released by 1 hour.

20. The method of treating of claim 19, wherein the dissolution rate of the ropinirole or salt thereof is between 5-10% by 1 hour.

21. The method of treating of claim 18, wherein the formulation has a dissolution rate wherein 95-100% is released by 36 hours.

22. The method of treating of claim 16, wherein the dissolution rate of the ropinirole or salt thereof is between 70-90% by 20 hours.

23. The method of treating of claim 16, wherein the formulation system comprises a monolayer tablet wherein the monolayer tablet comprises a substantially homogenous mixture of all components.

24. The method of treating of claim 16, wherein the formulation further comprises carboxymethylcellulose, hydrogenated castor oil, dibasic calcium phosphate and at least one diluent.

25. The method of treating of claim 16, wherein the formulation system comprises a pressed coating formulation.

26. The method of treating of claim 16, wherein the therapeutically effect amount of ropinirole is about 1.0 mg.

27. The method of treating of claim 16, wherein the therapeutically effect amount of ropinirole is about 12.0 mg.
